(19) United States
(12) Patent Application Publication
(10) Pub. No.: US 2014/0024630 A1
(43) Pub. Date: Jan. 23, 2014

(54) METHOD FOR THE NON-SURGICAL TREATMENT OF LIPODYSTROPHY IN A RETROVIRUS-INFECTED INDIVIDUAL

(71) Applicant: Raffaele RAUSO, Santa Maria Capua Vetere (Caserta) (IT)

(72) Inventor: Raffaele RAUSO, Santa Maria Capua Vetere (Caserta) (IT)

(73) Assignee: Pasquale MOTOLESE, San Giovanni in Marignano (Rimini) (IT)

(21) Appl. No.: 13/553,066

(22) Filed: Jul. 19, 2012

(51) Int. Cl. A61P 31/18; C07J 9/00; A61K 31/56

(52) U.S. Cl. 514/182; 552/553

(57) ABSTRACT

The present invention refers to a method for the non-surgical treatment of a lypodistrophy caused by an antiretroviral therapy in an individual infected by a retrovirus, such as HIV, and undergoing said antiretroviral therapy said method comprising the step of administering an effective amount of a adipocitolytic composition comprising deoxycholic acid and/or a pharmaceutically acceptable salt thereof in a retrovirus-infected individual affected by said lipodistrophy.

### FIELD OF THE INVENTION

The present invention is related to a method for the non-surgical treatment of a lipodystrophy caused by an antiretroviral therapy in an individual infected by a retrovirus, such as HIV, and undergoing said antiretroviral therapy.

### BACKGROUND OF THE INVENTION

The medical management of Human Immunodeficiency Virus (HIV) infection has really changed over the last years in view of both the improved knowledge of the infection pathogenesis and the new and emerging therapies for the clinical management of HIV infection.

Nowadays the standard HIV infection treatment, called “highly active antiretroviral therapy”, or HAART, combines three or more different drugs, such as two nucleoside reverse transcriptase inhibitors (NRTIs) and a protease inhibitor (PI), two NRTIs and a non-nucleoside reverse transcriptase inhibitor (NNRTI) or other such combinations. These drugs, by reducing the likelihood that the virus develops resistance, has the potential both to reduce mortality and morbidity rates among HIV-infected people, and to improve their quality of life.

Thus, with the advent of highly active antiretroviral therapy (HAART), HIV infection is now manageable as a chronic disease in patients who have access to medication and who achieve durable virologic suppression.

However, while these drugs work well in blocking viral infection, the drawback is that their side effects can be as bad as HIV infection itself.

For example, HIV-associated lipodystrophy is a syndrome that occurs in HIV-infected undertake antiretroviral medications, thus it represents a medication-associated condition. The term HIV-associated lipodystrophy refers to abnormal distribution of subcutaneous fat occurring in 40-80% of HIV-infected patients treated with antiretroviral therapy.

On the basis of the clinical features of HIV-associated lipodystrophy three main groups of patients can be identified: a subset of patients shows localized loss of fat tissue (lipoatrophy); another subset of patients has central fat accumulation (lipohypertrophy) such as dorsocervical fat accumulation (“buffalo hump”); and, finally, a subset of patients exhibit a mixed clinical picture.

The HIV-associated lipodystrophy clinical variants is also associated to glucidic and lipidic metabolic changes. Thus, a very wide range of morphologic and metabolic features always affect HIV-infected patients. The several clinical signs or stigmata of HIV-associated lipodystrophy, by worsening the already compromised physiological conditions of infected patients, can impair the effectiveness of the antiretroviral therapy.

The only currently available clinical interventions to treat

HIV-infected lipodystrophy and to help the infected patients in recovering their body image involve surgery.

In fact, only by surgery these patients reconstruct the harmonic relationship with the appearance of their own body that is severely compromised by both the virus and the drugs.

An aesthetic improvement even small can be really perceived as a big satisfaction by these patients.

Therefore, it would be desirable in this field to identify new therapeutic approaches to prevent, cure or ameliorate a lipodystrophy caused by or associated to an antiretroviral therapy in patients in need thereof.

This need is particularly desired in preventing, curing or ameliorating HIV-associated lipodystrophy to improve the life quality of infected patients that is already demanding and painful because of the viral infection.

### SUMMARY OF THE INVENTION

The present invention solves the above disclosed need by an adipocitolytic composition based on deoxycholic acid and/or a pharmaceutically acceptable salt thereof.

In fact, the Applicant has unexpectedly found good results in treating, by non surgical means, a lipodystrophy condition in an individual caused by or associated to an antiretroviral therapy by administering an effective amount of an adipocitolytic composition based on deoxycholic acid and/or a pharmaceutically acceptable salt thereof in an individual having a retrovirus infection, such as HIV infection, and undergoing an antiretroviral therapy.

This composition allows the destruction of adipocytes thus reducing fat tissue.

The Applicant has shown for the first time that a significant reduction in the thickness of the fat regions, especially the buffalo hump conditions, can be observed in an individual having a retrovirus infection, such as HIV infection, and undergoing an antiretroviral therapy following the application of the method according to the present invention.

The individuals undergoing to the non-surgical method according to the present invention have demonstrated a high degree of satisfaction with the results they reached.

A detailed description of the method for the non-surgical treatment of a retrovirus-associated lipodystrophy, in particular a buffalo hump condition, in an individual having a retrovirus infection and undergoing an antiretroviral therapy is reported as follows.

### BRIEF DESCRIPTION OF THE DRAWINGS

FIG. 1 depicts a lateral view of buffalo hump condition before (a) and after (b) three sits of treatment with the composition of the invention.

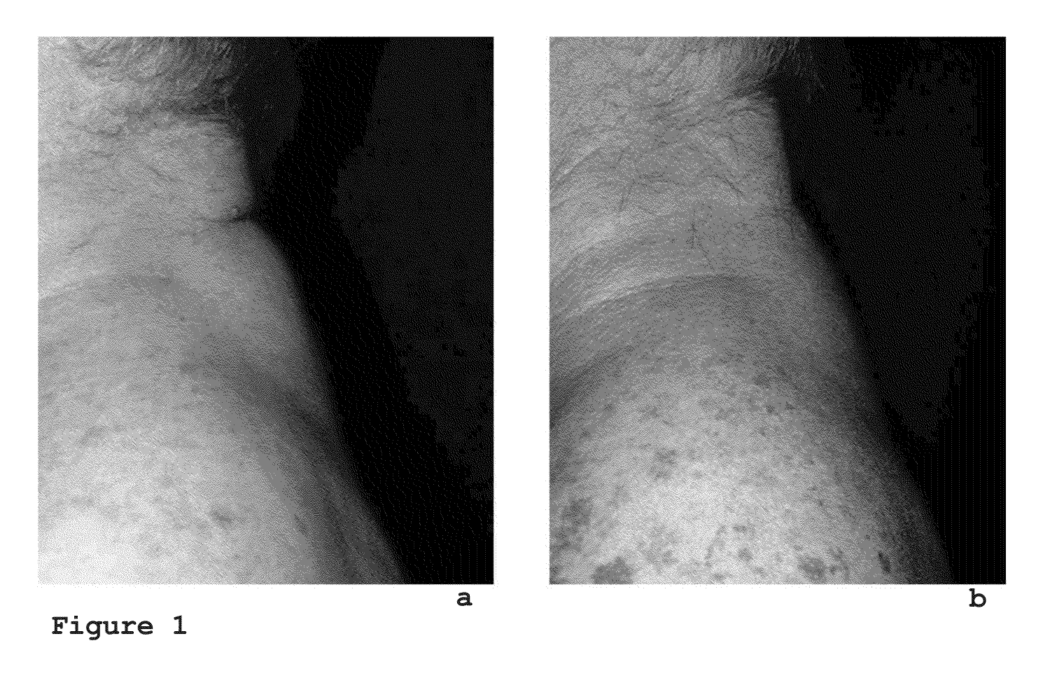

FIG. 2 depicts a frontal view of buffalo hump condition before (a) and after (b) three sits.

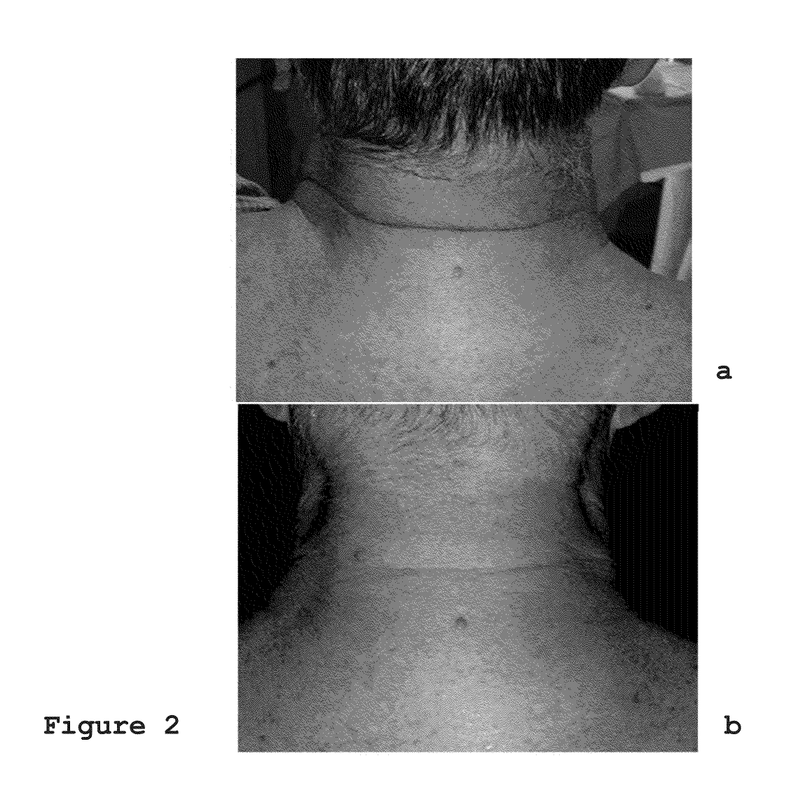

FIG. 3 depicts ultrasound measurements of subcutaneous thickness before (a) and after (b) three sits of treatment with the composition of the invention.

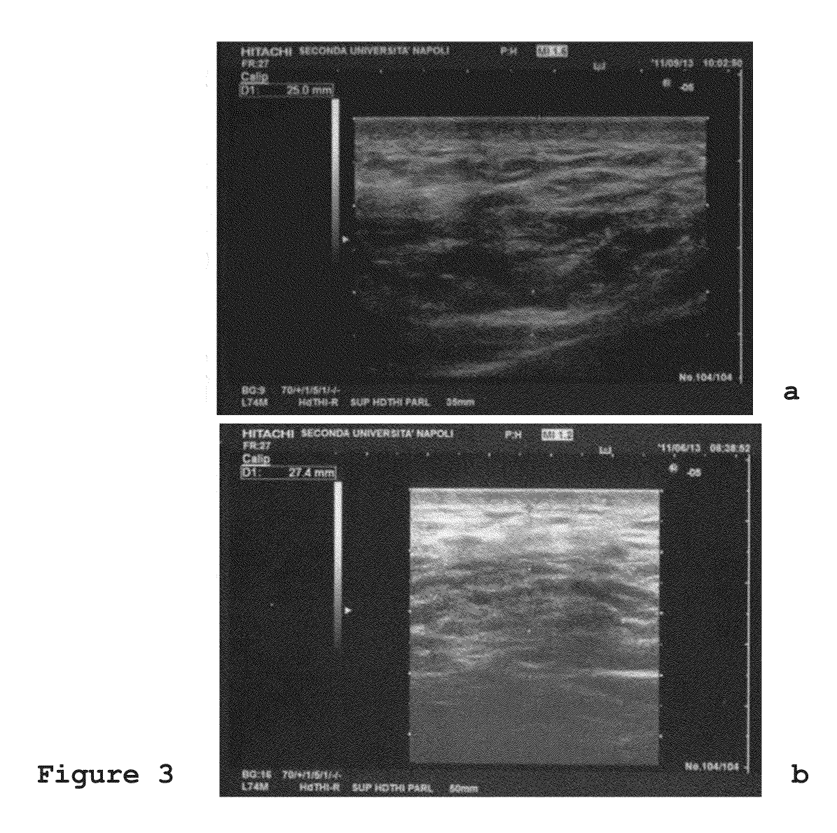

### DETAILED DESCRIPTION OF THE INVENTION

The method according to the present invention refers to the non-surgical treatment of a lipodystrophy (or lipodystrophic condition) caused by or associated to an antiretroviral therapy.

In particular, the lipodystrophy affects an individual infected by a retrovirus, such as HIV, and is caused by or associated to an antiretroviral therapy that the individual undertakes in order to treat the retroviral infection.

For the purpose of the present invention a lipodystrophy refers to a lipodistrophic condition, preferably the buffalo hump condition.

Preferably the buffalo hump condition of the present invention appears as a side effect in an individual having a retrovirus infection during or after an antiretroviral therapy performed by said individual to treat the retroviral infection.

In particular the retrovirus is Human Immunodeficiency Virus (HIV).

The method for the non-surgical treatment of retrovirus-associated lipodistrophy of the present invention comprises the administration of an effective amount of an adipocitolytic or lipolytic composition comprising deoxycholic acid and/or a pharmaceutically acceptable salt thereof to an individual infected by said retrovirus and underwent an antiretroviral therapy causing or associated to said retrovirus-associated lipodistrophy.

Alternatively, the present invention refers to an adipocitolytic or lipolytic composition for use in the non-surgical treatment of a retrovirus-associated lipodistrophy in a retrovirus-infected individual underwent an antiretroviral therapy said adipocitolytic composition comprising deoxycholic acid and/or a pharmaceutically acceptable salt thereof. In particular, said deoxycholic acid and/or a pharmaceutically acceptable salt thereof is/are administrable in an effective amount to said individual infected by a retrovirus and underwent an antiretroviral therapy.

According to a preferred embodiment of the present invention, the administered adipocitolytic composition comprises the sodium salt of deoxycholic acid, preferably (3α,5β,12α,20R)-3,12-dihydroxy-5colan-24-oic acid sodium salt.

Said deoxycholic acid and/or a pharmaceutically acceptable salt thereof is preferably present in the composition in a concentration ranging from 0.1 to 10 mg/ml, preferably 0.3-6 mg/ml, more preferably 0.5-5 mg/ml.

According to another preferred embodiment of the present invention the adipocitolytic composition comprises deoxycholic acid and/or a pharmaceutically acceptable salt thereof and further at least a polymer. Preferably said deoxycholic acid salt is a sodium salt, more preferably a (3α,5β,12α,20R)-3,12-dihydroxy-5colan-24-oic acid sodium salt. Preferably said at least a polymer is a polymer of 3,6-anhydro-L-galattose and D-galattose.

According to another preferred embodiment of the present invention the adipocitolytic composition comprises deoxycholic acid and/or a pharmaceutically acceptable salt thereof, at least a polymer and further at least a buffer. Preferably said deoxycholic acid salt is a sodium salt, more preferably a (3α,5β,12α,20R)-3,12-dihydroxy-5colan-24-oic acid sodium salt. Preferably said at least a polymer is a polymer of 3,6-anhydro-L-galattose and D-galattose. Said buffer is selected from the group consisting of: phosphate buffer, carbonate buffer and aminoacidic buffer.

For the purpose of the present invention the preferred buffer is phosphate buffer. More preferably said buffer is used in an amount needed to determine a pH of the composition ranging from 6-8.

According to another preferred embodiment of the present invention the adipocitolytic composition further comprises at least one pharmacologically acceptable excipient, in other words a compound useful in the preparation of the adipocitolytic composition that is biologically safe and atoxic.

Moreover, the adipocitolytic composition can further comprises: a lubricant agent; a humectant agent; an emulsifying and/or suspending agent; a preserving agent; a dispersing agent or a molecule facilitating tissue penetration.

According to another embodiment of the present invention, the adipocitolytic composition is preferably formulated as injectable and/or deliverable solution, preferably for the topic treatment of the lipodistrophy in the infected individual.

Alternatively the adipocitolytic composition is formulated for topical use as a cream, a gel, an oil, an emulsion or an ointment.

The adipocitolytic composition can be further formulated for releasing the active ingredient immediately or in a controlled manner after the administration.

For the purpose of the present invention the preferred formulation of the adipocitolytic composition is an injectable and/or delivering solution. Preferably, the injectable solution is an aqueous solution, more preferably is an aqueous micro-gelatinous solution. In particular, the injectable solution comprises water and/or sodium chloride and/or a further pharmaceutically acceptable delivering agent.

The adipocitolytic solution in an injectable form is preferably topically injected into the accumulated fat area, such as the dorsocervical region if buffalo hump is present in the infected individual.

In particular, the injection is performed by using at least one intralipotherapy needle.

According to a preferred embodiment of the present invention 5-8 milliliters of the adipocitolytic composition of the present invention is injected, preferably topically injected into the area to be treated.

The injection of the adipocitolytic solution is performed in one or more sits, preferably at least three sits.

In general, the number of sits is related to the degree of fat accumulation to be treated.

The injection sits can be scheduled every fifteen days, preferably one per month.

According to a preferred embodiment of the present invention, the method of treatment is preceded, accompanied or followed by a treatment, preferably a topical treatment, with ultrasounds.

Alternatively the method according to the present invention can be associated with other protocol for treating a lipodistrophy, preferably said protocol is selected from the group consisting of: administration of (or exposure to) external or internal ultrasounds; administration of (or exposure to) radio frequencies; laser administration (or exposure) and carboxytherapy. Moreover said protocol can be used in combination with or alteratively to: ultrasouds, ionophoresys, electrophoresis, radio frequencies, cryophoresis, cutaneous occlusion techniques and/or active or passive cutaneous absorbiment.

A further aspect of the present invention refers to a kit for performing the method for the non-surgical treatment of lipodistrophya in an individual infected by retrovirus, said kit comprising the adipocitolytic composition according to the detail description of the present invention.

In particular, the adipocitolytic composition is an aqueous micro-gelatinous injectable solution.

More preferably the adipocitolytic solution is in disposable tubes, such as vials, preferably predosed, useful for each treatment sit.

According to a preferred embodiment f the present invention said kit further comprises at least one needle, preferably at least one intralipotherapy needle.

### EXAMPLES

The individual treated with the method according to the present invention is a 52-years old Caucasian man, HIV positive in HAART (HAART—Highly Active AntiRetroviral Therapy) treatment for 12 years.

The individual shows lipodistrophya as antiretroviral therapy side effect. In particular, he shows facial lipoatrophy and a buffalo hump deformity.

Therefore, buffalo hump deformity of said individual has been cured by injecting the aqueous micro-gelatinous lipolithic solution Aqualyx® after he has signed the informed consent.

Before buffalo hump treatment, an ultrasonographic examination, and photographic documentation of dorsocervical fat pad has been performed. Three sits have been performed, one per month; in each sit a vial of 8 ml of Aqualyx® has been injected using intralipotherapy large area needles (Lipoinject® series) as explained by the producer.

At each sit the area to be injected has been carefully cleaned with clorexidine 0.2%; sterile gloves have been used by the physician, asepsis rules have been followed; no antibiotic therapy has been performed.

After each injection session, a well tolerated light swelling and ecchymosis have been referred by the individuals which gradually disappeared in about five days.

Neither major nor minor complications have been registered.

Clinical improvement has been documented by photos and has been reported by the patient after each treatment.

Two weeks after last sit a new ultrasonographic examination has been performed to assess the result. It has been showed 2.4 mm reduction of the dorsocervical fat pad.

Therefore the results have clearly demonstrated that the administration of a solution of Aqualyx® containing short half life detergents is a safe and efficient method for buffalo hump reduction and it can be used in substitution of or as an alternative of the more invasive surgery treatment.

1. A method for the non-surgical treatment of a retrovirus-associated lipodistrophy caused by or associated with an antiretroviral therapy in a retrovirus-infected individual said method comprising the step of administering an effective amount of an adipocitolytic composition comprising deoxycholic acid and/or a pharmaceutically acceptable salt thereof and at least a polymer of 3,6-anhydro-L-galattose and D-galattose to said retrovirus-infected individual affected by said lipodistrophy.

2. The method according to claim 1, wherein said lipodistrophy is a buffalo hump.

3. The method according to claim 1, wherein said retrovirus is Human Immunodeficiency Virus (HIV).

4. The method according to claim 1, wherein said adipocitolytic composition comprises (3α,5β,12α,20R-3,12-dihydroxy-5colan-24-oic acid sodium salt.

5. The method according to claim 1, wherein said deoxycholic acid and/or a pharmaceutically acceptable salt thereof is present in a concentration ranging from 0.1 to 10 mg/ml.

6-7. (canceled)

8. The method according to claim 1, wherein said adipocitolytic composition further comprises at least one buffer.

9. The method according to claim 6, wherein said at least one buffer is selected from the group consisting of: phosphate buffer, carbonate buffer and aminoacidic buffer.

10. The method according to claim 9, wherein said buffer is phosphate buffer.

11. The method according to claim 1, wherein said adipocitolytic composition further comprises at least one pharmacologically acceptable excipient.

12. The method according to claim 1, wherein said adipocitolytic composition further comprises: a lubricant agent; a humectant agent; an emulsifying and/or suspending agent; a preserving agent; a dispersing agent or a molecule facilitating tissue penetration.

13. The method according to claim 1, wherein said adipocitolytic composition is formulated as injectable and/or delivering solution.

14. The method according to claim 13, wherein said injectable solution is an aqueous solution.

15. The method according to claim 13, wherein said injectable and/or delivering solution comprises water and/or sodium chloride and/or other pharmaceutically acceptable delivering agents.

16. The method according to claim 13 for the topical treatment of said retrovirus-associated lipodistrophy caused by or associated with said antiretroviral therapy.

17. The method according to claim 16, wherein said topical. treatment is performed by using at least one intralipotherapy needle.

18. The method according to claim 1, wherein said method is preceded, accompanied or followed by a treatment, preferably a topical treatment, with ultrasounds.

19. A kit for performing the method for non-surgical treating a lipodistrophy according to claim 1 said kit comprising at least one vial comprising a adipocitolytic composition comprising deoxycholic acid and/or a pharmaceutically acceptable salt thereof and at least a polymer of 3,6-anhydro-L-galattose and D-galattose.

20. The kit according to claim 19 further comprising at least one intralipotherapy needle.

21. The method according to claim 10, wherein said buffer has a pH ranging from 6-8.

22. The method according to claim 13, wherein said injectable solution is an aqueous microgelatinous solution.
